(19) United States
(12) Patent Application Publication
(10) Pub. No.: US 2011/0098492 A1
(43) Pub. Date: Apr. 28, 2011

(54) CLEANING COMPOSITIONS CONTAINING MID-RANGE ALKOXYLATES

(76) Inventors: Pierre T. Varineau, Lake Jackson, TX (US); Molly I-chin Busby, Midland, MI (US); Robert Kirk Thompson, Lake Jackson, TX (US); Thomas C. Eisenschmid, Cross Lnes, WV (US)

(21) Appl. No.: 13/000,250

(22) PCT Filed: Dec. 23, 2009
(86) PCT No.: PCT/US2009/046916

§ 371 Date: Dec. 20, 2010

Related U.S. Application Data
(60) Provisional application No. 61/073,522, filed on Jun. 18, 2008.
(60) Provisional application No. 61/140,095, filed on Dec. 23, 2008.

(51) Int. Cl. C07D 303/02

(52) U.S. Cl. 549/554

(57) ABSTRACT

Cleaning compositions are described comprising mid-range alkoxylate surfactants or blends of alkoxylate surfactants, and their use as cleaners for triglycerides and cross-linked triglycerides, formula stabilization agents, agents for ultra-concentrated cleaning formulations, pre-wash spotters, detergents, agricultural adjuvants, hard surface cleaning, and emulsifiers.

### FIELD

The present invention relates to cleaning compositions and surfactant manufacture.

### BACKGROUND

Currently, there is a strong market preference for surfactants that are readily biodegradable and environmentally acceptable. While alkylphenol ethoxylates (APEs) are widely recognized as outstanding surfactants in a broad variety of applications, including laundry, hard surface cleaning, paints and coatings, emulsification, and agricultural adjuvants, they do suffer from a poor public perception of their environmental compatibility.

Previously contemplated APE-replacement surfactants generally may have good performance profiles in a select few applications, but not in a broad variety of applications. For example, the biodegradable linear C12-16 primary alcohol ethoxylates work well in laundry, but they perform poorly in other applications such as hard surface cleaning or freeze-thaw stabilization for paints and coatings. One particular problem of interest is that many environmentally acceptable surfactants are ineffective on triglyceride and oxidatively cross-linked triglyceride soils, a particular set of difficult-to-clean soils which can form a hard varnish on pans, hoods, oven surfaces, and food preparation surfaces. Also, many previously contemplated APE-replacement surfactants are biodegradable, but not environmentally acceptable, or vice versa.

Thus, what is needed are surfactants that are effective, biodegradable, environmentally acceptable, alternatives to APEs for cleaning.

### SUMMARY

In one embodiment, the present invention provides cleaning compositions, comprising at least one nonionic surfactant represented by formula (I):

R¹—O—[(CH₂CH(R²)—O)ₓ(CH₂CH₂O)_{y}]_{z}—H (I)

wherein x is, independently at each occurrence, 0 or a real number from about 1 to about 11, provided that, in at least one occurrence, x is greater than 0; y is, independently at each occurrence, 0, or a real number from about 1 to about 20, provided that, in at least one occurrence, y is greater than 0; z is a whole number between 1 and 50; R¹ is a C₆₋₁₀ branched or linear alkyl; and R² is CH₃ or CH₂CH₃.

In another embodiment, the present invention provides methods of removing cross-linked triglycerides from a surface, comprising applying the present cleaning compositions to the surface.

In yet another embodiment, the present invention provides methods of preparing a nonionic surfactant from an octene purge stream, comprising obtaining the unreacted internal octenes after reacting ethylene with 1-octene; converting the internal octenes to alcohols; and reacting the alcohols with a block of propylene oxide or butylene oxide, followed by a block of ethylene oxide; thereby forming a nonionic surfactant represented by formula (II):

R¹—O—(CH₂CH(R²)—O)ₓ(CH₂CH₂O)_{y}—H (II)

wherein x is a real number from about 1 to about 11; y is a real number from about 1 to about 20; R¹ is a C₆₋₁₀ branched or linear alkyl; and R² is, independently at each occurrence, CH₃ or CH₂CH₃.

### DESCRIPTION

In one embodiment, the present invention provides cleaning compositions comprising mid-range alkoxylate surfactants or blends of alkoxylate surfactants, and their use as cleaners for triglycerides and cross-linked triglycerides, formula stabilization agents, agents for ultra-concentrated cleaning formulations, pre-wash spotters, detergents, agricultural adjuvants, hard surface cleaning, and emulsifiers.

In one embodiment, the present invention provides cleaning compositions, comprising at least one nonionic surfactant represented by formula (I):

R¹—O—[(CH₂CH(R²)—O)ₓ(CH₂CH₂O)_{y}]_{z}—H (I)

wherein x is, independently at each occurrence, 0 or a real number from about 1 to about 11, provided that, in at least one occurrence, x is greater than 0; y is, independently at each occurrence, 0, or a real number from about 1 to about 20, provided that, in at least one occurrence, y is greater than 0; z is a whole number between 1 and 50; R¹ is a C₆₋₁₀ branched or linear alkyl; and R² is, independently at each occurrence, CH₃ or CH₂CH₃.

It is understood that “x” and “y” represent average degrees of, respectively, propoxylation and/or butoxylation (depending on the identity of R²) and ethoxylation. Thus, x and y need not be whole numbers, which is intended to be illustrated by use of “about.” Taken together, x and y establish a degree of alkoxylation in an oligomer distribution. It should be apparent that the order of x and y is block or random, with x being the first and/or last block.

Likewise, “z” is a whole number, as it represents the number of iterations of the formula. For example, for a PDₓ-EO_{y}-BOₓ oligomer, z would be 2 and the second y would be zero. For a EO_{y}-BOₓ-POₓ-oligomer, z would be 3, with the first x and the second and third y's zero.

R¹ is a branched or linear alkyl that results when the corresponding branched or linear alcohol compound is alkoxylated. Methods for making the nonionic surfactants of the invention by the alkoxylation of alcohols are discussed below. R¹ can be any C₆₋₁₀ branched or linear alkyl.

The composition may further include co-formulation additives such as water, co-surfactants, anionic surfactants, cationic surfactants, amine oxides, alkyl amine oxides, solvents, chelating agents, bases such as monoethanolamine, diethanolamine, triethanolamine, potassium hydroxide, sodium hydroxide, or other bases, and other conventional formulation ingredients.

In a preferred embodiment, the nonionic surfactant is represented by formula (II):

R¹—O—(CH₂CH(R²)—O)ₓ)(CH₂CH₂O)_{y}—H (II)

wherein x is a real number from about 1 to about 11; y is a real number from about 1 to about 20; R¹ is a C₆₋₁₀ branched or linear alkyl; and R² is CH₃ or CH₂CH₃.

In one embodiment, x is preferably about 4, 5, or 6, most preferably about 5.

In one embodiment, y is preferably about 3, 6, 9, or 11, most preferably about 6.

R¹ can be any C₆₋₁₀ branched or linear alkyl, however in a preferred embodiment, R¹ is a C₈₋₉ branched alkyl. In one embodiment, R¹ is R1 is 2-ethylhexyl or 2-propylhexyl, preferably 2-ethylhexyl.

In one embodiment, R¹ is derived from alcohols that are produced from internal octenes. “Internal octenes” refers to the unreacted residual, or byproduct, left behind when reacting ethylene with 1-octene to produce ethylene/1-octene copolymers (“EOC's”). These internal octenes can be obtained as a purge stream from the process, and then can be converted to alcohols by a process which will be described hereinafter. Alcohols produced from internal octenes include at least one of 1-nonanol, 2-methyl-1-octanol, 2-ethyl-1-septanol, 2-propyl-1-hexanol, 3-methyl-4-hydroxymethyl septane, 3-methyl-3-hydroxymethyl-septane, or 2-hydroxymethyl-3-methyl septane. Normally, the alcohols will be a blend, depending on the source of the 1-octene.

In one embodiment, R² is CH₃, thus representing a propylene oxide. In other embodiments, R² is CH₂CH₃, thus representing a butylene oxide.

Preferred surfactants of Formula II are those wherein x is about 4, 5, or 6; y is about 3, 6, 9, or 11; R¹ is a C₈₋₉ branched alkyl, and R² is CH₃. Most preferred surfactants of Formula II are those wherein wherein x is 5; y is 6; R¹ is 2-ethyl hexyl, and R² is CH₃. Preferably, the PO or BO portion, and EO portion are the result of a block feed.

Applicants surprisingly have found that the above-described surfactants exhibit the ability to clean cross-linked triglycerides as well as APEs (i.e., nonylphenoxy (polyoxyethylene-9) (“NP-9”)). In addition, the claimed surfactants also have an acceptable environmental profile in that they are considered readily biodegradable according to OECD 301-series criterion, and also have an aquatic toxicity of greater than 10 mg/L.

### Methods of Making

The alcohols may be converted to alcohol alkoxylates by methods such as those discussed in “Nonionic Surfactants”, Martin, J. Schick, Editor, 1967, Marcel Dekker, Inc., or United States Patent Application Publication (USPAP) 2005/0170991A1 which is incorporated herein by reference in its entirety. Fatty acid alcohols may also be alkoxylated using metal cyanide catalysts including (but not limited to) those described in United States Patent Number (USP) U.S. Pat. No. 6,429,342.

Alkoxylation processes may be carried out in the presence of acidic or alkaline catalysts. It is preferred to use alkaline catalysts, such as hydroxides or alcoholates of sodium or potassium, including NaOH, KOH, sodium methoxide, potassium methoxide, sodium ethoxide and potassium ethoxide. Base catalysts are normally used in a concentration of from 0.05 percent to about 5 percent by weight, preferably about 0.1 percent to about 1 percent by weight based on starting material. In one non-limiting embodiment, a C8 olefin mixture is first converted to an alcohol as described hereinabove, and subsequently converted to form a nonionic surfactant via alkoxylation with from greater than about 2 to about 5 moles of propylene oxide and from greater than about 1 to about 10 moles of ethylene oxide.

The addition of alkylene oxides may, in one non-limiting embodiment, be carried out in an autoclave under pressures from about 10 psig to about 200 psig, preferably from about 60 to about 100 psig. The temperature of alkoxylation may range from about 30° C. to about 200° C., preferably from about 100° C. to about 160° C. After completion of oxide feeds, the product is typically allowed to react until the residual oxide is less than about 10 ppm. After cooling the reactor to an appropriate temperature ranging from about 20° C. to 130° C., the residual catalyst may be left unneutralized, or neutralized with organic acids, such as acetic, propionic, or citric acid. Alternatively, the product may be neutralized with inorganic acids, such as phosphoric acid or carbon dioxide. Residual catalyst may also be removed using ion exchange or an adsorption media, such as diatomaceous earth. In many non-limiting embodiments the resulting alkoxylated material may be an effective surfactant.

The final poly(alkylene oxide) capped poly(alkylene oxide)-extended linear or branched alcohol of the invention may be used in formulations and compositions in any desired amount. However, it is commonly known to those skilled in the art that levels of surfactant in many conventional applications may range from about 0.05 to about 90 weight percent, more frequently from about 0.1 to about 30 weight percent, and in some uses from about 0.5 to about 20 weight percent, based on the total formulation. Those skilled in the art will be able to determine usage amounts via a combination of general knowledge of the applicable field as well as routine experimentation where needed.

### Biodegradability and Environmental Acceptability

A global standard screening test for the aerobic biodegradation of surfactants is based on the Organization for Economic Cooperation and Development (OECD) 301 28-day modified Sturm test, which gives results as “readily biodegradable” (>=60% biodegradation) “inherently biodegradable” (>=20% but less than 60%) or “non biodegradable” (<20%). For global regulatory compliance, it is broadly perceived that any new surfactants developed and commercialized should meet the “readily biodegradable” classification using the OECD 301 series aerobic tests.

In addition to meeting the status of “readily biodegradable”, surfactants should also have an acceptable aquatic toxicity. Guidelines set by the “Design for the Environment (DfE) require that surfactants have an aquatic toxicity of greater than 10 milligrams/liter to be classified as DfE compliant.

Short-chain surfactants commonly used in hard surface cleaning, such as the undecanol-based NEODOL™ 1-5 or 1-9, or the 2-Propyl Heptanol based LUTENSOL™ XP- or XL-series are not as effective as APEs in the cleaning of triglycerides or cross-linked triglycerides and, in some cases, also do not pass the DfE criteria.

Longer-chain, highly branched surfactants, such as the TERGITOL™ Trimethyl Nonanol-6 (TMN-6) shows good performance cleaning of triglycerides or cross-linked triglycerides, however, these longer-chain, highly branched surfactants are not biodegradable.

In one embodiment, the surfactant is readily biodegradable using OECD 301 F testing methodology (defined by greater than 60% biodegradation), and exhibits an aquatic toxicity of greater than 10 mg/L for Daphnia and Algae according to the following tests: Organization for Economic Cooperation and Development (OECD): OECD Guidelines for the Testing of Chemicals, “Freshwater Alga and Cyanobacteria, Growth Inhibition Test”, Procedure 201, adopted 23 Mar. 2006; European Economic Community (EEC): Commission directive 92/69/EEC of 31 Jul. 1992, Methods for the determination of ecotoxicity, C.3., “Algal Inhibition Test”.

OECD Guidelines for the Testing of Chemicals, “Freshwater Alga and Cyanobacteria, Growth Inhibition Test”, Procedure 201, adopted 23 Mar. 2006; European Economic Community (EEC): Commission directive 92/69/EEC of 31 Jul. 1992, Methods for the determination of ecotoxicity, C.3., “Algal Inhibition Test”.

### Formulation Stability

In addition to the lack of effective alternatives to APE's for the cleaning of cross-linked triglycerides, another challenge facing the surfactants industry is formula stability.

Concentrated formulas containing surfactants, solvents, builders (such as sodium citrate), chelating agents, and other ingredients are often not stable, and will separate out over time. In some cases, the phase separation causes a cloudy solution. In other cases, the phase separation causes multiple liquid layers to form, such as a top layer and bottom layer. Phase separation can be a significant problem for consumers, because the performance of the phase-separated product is often not as good as the homogeneous product. Often, once phase separation occurs, it is difficult or impossible to get the formulation back to a homogeneous state.

Formulas are typically stabilized through the addition of hydrotropes, such as sodium xylene sulfonate (SXS) or the phosphate ester of ethoxylated cresylic acid, or the phosphate esters of ethoxylated alcohols, or through the addition of other hydrotropes. Hydrotropes typically do not add any other function to the formula, other than to stabilize the components and to prevent phase separation. In particular, they do not significantly reduce surface tension, so they are not effective surfactants.

The concept of a “multi-functional” compound is one in which a formulation ingredient offers several functions within a formula. A “surface active hydrotrope”, is a compound that acts as both a hydrotrope and a surfactant. This type of a multifunctional compound would enable formulators to create stable formulas without the addition of hydrotrope, and thus greatly simplify the creation of stable formulas.

Applicants have surprisingly found that the presently claimed surfactants act as hydrotroping agents, and are capable of stabilizing formulations in the absence of hydrotropes. These C6-C10 alkoxylates are multi-functional, acting as both a surfactant and a hydrotrope.

### Concentrates

A recent trend promotes production of ultra-concentrated formulations or systems that contain little or no water. Such formulations or concentrates are delivered to an end-use customer who then dilutes the concentrate with water to produce a final working solution. Those who use concentrates consider it an eco-friendly approach as it eliminates costs associated with shipping water and reduces material requirements for packaging. The concentrates typically include one or more nonionic surfactants because they are compatible with all other surfactant types (e.g. anionic, cationic and zwitterionic surfactants). In addition, nonionic surfactants resist precipitation with hard water and offer excellent oil grease cleaning benefits.

Household and industrial applications that employ ultra-concentrates include laundry detergents, hard surface cleaners, automatic dishwasher detergents, rinse aids, emulsification packages (such as agricultural-emulsifiers), and flotation systems (for applications such as paper de-inking and ore flotation).

Soap and detergent manufacturers use the term “diluted” to refer both to dissolution of solids and reduction of concentration of liquids. For example, liquid laundry detergent may be diluted in a tub of water. Similarly, a powdered or block laundry detergent that is dissolved in a tub of water also would be referred to as “diluted.”

A common problem for concentrated formulas that contain surfactants is formation of gels when a solid or liquid surfactant is diluted with water. For example, a formulation or concentrate consisting primarily of a 9-mole ethoxylate of nonylphenol (such as TERGITOL™ NP-9) forms resilient, slow-dissolving gels when mixed with water. For end-use customers (especially household customers), these slow-dissolving gels require extensive mixing which can interfere with convenience and effectiveness of end-use or diluted formulations.

One way the industry expresses a tendency of a surfactant to cause gels is a “gel range.” A typical gel range describes a percentage of samples that form gels, out of a number of samples, each having increasing surfactant concentration. For example, a gel range of less than 20% indicates that less than two samples out of nine samples form gels; the nine samples having surfactant concentrations of 10 wt. %, 20 wt. %, 30 wt. %, 40 wt. %, 50 wt. %, 60 wt. %, 70 wt. %, 80 wt. %, and 90 wt. %, each weight percentage (wt %) being based upon combined weight of surfactant and de-ionized water. A sample forms a gel when it is non-pourable for at least five seconds at 23° centigrade (° C.) when its container is inverted 180° so the container's open spout or mouth faces down. For many applications, a surfactant ideally has no gel range. In other words, it does not form gels when mixed with water.

In some cases, the tendency to form gels can be overcome by adding an anti-gelling agent such as a solvent or a polyglycol to the formulation. For example, a simple formulation containing 20 wt % of a 9-mole ethoxylate of nonylphenol (Tergitol™ NP-9) and 80 wt % propylene glycol (each wt % based on formulation weight) will not form gels upon dilution with water. However, the addition of anti-gelling agents tends to increase overall complexity and cost of the formulation, and therefore may be undesirable.

In one embodiment, the presently claimed surfactants exhibit a gel range less than 20% of the range from 0% to 100%, when blended with water.

In addition to gel formation tendency, an important physical property consideration for use in selecting a surfactant is its tendency to undergo a viscosity increase as temperatures fall or decrease. Surfactant users typically select “pour point” or “pour point temperature” as a general indicator of handling characteristics of a pure surfactant under reduced temperatures. They consider pour point as that temperature below which a liquid surfactant will fail to pour from a container.

Relatively short-chain alkoxylates of linear alcohols derived from petroleum or natural gas, for example, TRITON™ XL-80N, based on an alkoxylate of a C₈-C₁₀ blend of alcohols, PLURAFAC™ SLF-62 (based on a C₆₋₁₀ alkoxylate blend), ALFONIC™ 810-60 (a C₈-C₁₀ ethoxylate), and SURFONIC™ JL-80X (a C₈₋₁₀ alkoxylate) do exhibit a narrow gel range, but perform poorly as alternatives to APEs for the cleaning of triglyceride and cross-linked triglyceride soils.

### Sustainability

There is always an interest in producing useful chemicals from by-products. As mentioned above, in one embodiment, R¹ is an alkyl that is derived from an alcohol produced from internal octenes, the unreacted residual, or byproduct, left behind when reacting ethylene with 1-octene.

In one embodiment, the present invention provides methods of preparing a nonionic surfactant from an octene purge stream, comprising: obtaining the unreacted internal octenes after reacting ethylene with 1-octene; converting the internal octenes to alcohols; and reacting the alcohols with a block of propylene oxide or butylene oxide, followed by a block of ethylene oxide; thereby forming a nonionic surfactant represented by formula (II):

R¹—O—(CH₂CH(R²)—O)ₓ(CH₂CH₂O)_{y}—H (II)

wherein x is a real number from about 1 to about 11; y is a real number from about 1 to about 20; R¹ is a C₆₋₁₀ branched or linear alkyl; and R² is CH₃ or CH₂CH₃.

Suitable nonanols may be derived from a blend of octenes via the OXO Process wherein the mixture is treated by hydroformylation. Blends of 1-octene with internal octenes are a common by-product of the ethylene-octene co-polymerization process practiced by plastics producers worldwide. Hydroformylation is defined as a reaction that involves adding hydrogen and carbon monoxide across a double bond to yield aldehyde products. In this particular functionalization of the by-product mixture, a subcategory of hydroformylation, referred to as the OXO process, involves treating the by-product mixture with a combination of hydrogen and carbon monoxide in the presence of a catalyst based on rhodium or another transition metal, such as cobalt, platinum, palladium, or ruthenium. The hydroformylation catalyst may be of homogeneous or heterogeneous type. Such catalysts may be prepared by methods well known in the art. In certain embodiments the catalyst for this hydroformylation is a metal-ligand complex catalyst.

In certain embodiments the metals which are included in the metal-ligand complex catalyst include Groups 8, 9 and 10 metals selected from rhodium (Rh), cobalt (Co), iridium (Ir), ruthenium (Ru), iron (Fe), nickel (Ni), palladium (Pd), platinum (Pt), osmium (Os), and mixtures thereof, with the preferred metals being palladium, rhodium, cobalt, iridium and ruthenium, more preferably palladium, rhodium, cobalt and ruthenium, and in certain particular and non-limiting embodiments, palladium. The ligands may include, for example, organophosphorus, organoarsenic and organoantimony ligands, and mixtures thereof, and in certain non-limiting embodiments organophosphorus ligands may be selected. These may include organophosphines, e.g., mono-, di-, tri- and poly-(organophosphines), and organophosphites, e.g., mono-, di-, tri- and poly-(organophosphites). Other suitable organophosphorus ligands may include, for example, organophosphonites, organophosphinites, amino phosphines and the like. Other suitable ligands include, for example, heteroatom-containing ligands, such as 2,2′-bipyridyl and the like. In some non-limiting embodiments rhodium-based metal-ligand complex catalysts which employ phosphorus based ligands or mixtures of ligands may be selected. In other non-limiting embodiments mixtures of such catalysts may be selected.

The concentrations of complexed ligand, metal, and catalyst in general in the hydroformylation reaction will depend upon selected constituents, reaction conditions and solvent employed. For example, in some embodiments the concentration of complexed ligand may range from about 0.005 to about 25 weight percent, based on total weight of the reaction mixture. In other particular and non-limiting embodiments, the complexed ligand concentration may range from about 0.01 to about 15 weight percent, and preferably from about 0.05 to about 10 weight percent, based on total weight of the reaction mixture. In general, the concentration of the metal may be from a few parts per million by weight to as high as about 2000 parts per million by weight or greater, based on the weight of the reaction mixture. In certain particular and non-limiting embodiments, the metal concentration may range from about 50 to about 1500 parts per million by weight, based on the weight of the reaction mixture, and more preferably is from about 70 to about 1200 parts per million by weight, based on the weight of the reaction mixture. Thus, the molar ratio of complexed ligand:metal may, in fact, range from about 0.5:1 to about 1000:1. In some non-limiting embodiments the overall concentration of catalyst in the reaction mixture may range from several parts per million to several percent, based on weight of the reaction mixture.

In addition to the metal-ligand complex catalyst, free ligand (i. e., ligand that is not complexed with the metal) may also be present in the hydroformylation reaction mixture. The free ligand may correspond to, for example, any of the ligands discussed hereinabove as employable herein. It is in some embodiments preferred that the free ligand be the same as the ligand of the metal-ligand complex catalyst employed, but such is not required. The hydroformylation reaction may involve up to 100 moles, or more, of free ligand per mole of metal in the hydroformylation reaction mixture. Preferably the hydroformylation reaction is carried out in the presence of from about 0.25 to about 50 moles of coordinatable phosphorus, and more preferably from about 0.5 to about 10 moles of coordinatable phosphorus per mole of metal present in the reaction medium, with the amounts of coordinatable phosphorus being the sum of both the amount of coordinatable phosphorus that is bound (complexed) to the palladium metal present and the amount of free (non-complexed) coordinatable phosphorus present. If desired, make-up or additional coordinatable phosphorus may be supplied to the reaction mixture at any time and in any suitable manner, for example, to maintain a predetermined level of free ligand in the reaction mixture.

The OXO process may be accomplished effectively, in certain non-limiting embodiments, under relatively high pressures (from subatmospheric to about 100 atmospheres) and at temperatures from about 40° C. to about 300° C., but a wider range of temperatures from about 10° C. to about 400° C. and pressures from about 10 psig to about 3000 psig may be employed, provided that the desired end result is achieved. This result is production of a mixture of aldehydes, each of which has one more carbon atom than the specific C10-C20 olefin from which it was made.

The product aldehydes may be separated from the hydroformylation mixture by conventional means such as vaporization or distillation. The aldehyde products may also be separated from the hydroformylation catalyst by phase separation. An example of such is where a phosphorus based ligand has been designed to preferentially phase separate into a polar or aqueous-polar phase, and consequentially the metal, e.g., rhodium, and ligand components may be readily recovered from the relatively non-polar aldehyde product mixture. Such aldehydes may be useful as surfactants themselves or as hydrophobes therefor, or they may be subjected to further processing to produce derivatives as discussed hereinbelow.

Such further processing may involve treatment of the mixture of aldehydes with hydrogen over a suitable hydrogenation catalyst to form the corresponding alcohols. Because the feed involves a mixture of olefins, the result will be a mixture of alcohols. This hydrogenation may be carried out using a variety of known hydrogenation catalysts in conventional amounts. Such catalysts may be homogeneous or heterogeneous in type, and may comprise a variety of metals, including but not limited to palladium, ruthenium, platinum, rhodium, copper chromite, nickel, copper, cobalt, other Groups 8, 9 and 10 metals, chromium oxide, a variety of metal nitrides and carbides, combinations thereof, and the like. These metal catalysts may be supported on a variety of supports, including titania, magnesium silicate, lanthanum oxide, ceria, silicon carbide, magnesium silicate, aluminas, silica-aluminas, vanadia, combinations thereof, and the like. The catalysts may be further promoted by additional metals or other additives, including, but not limited to, barium, manganese, zirconium, selenium, calcium, molybdenum, cobalt, other Groups 8, 9 and 10 metals, copper, iron, zinc, combinations thereof, and the like. A variety of homogeneous catalysts may also be employed, comprising, for example, rhodium, ruthenium, cobalt, nickel and the like. Such catalysts may be promoted or stabilized by a variety of ligands including nitrogen or phosphorus containing materials such as, but not limited to, amines, phosphines, phosphites, combinations thereof, and similar materials. Those skilled in the art will understand that any catalyst that is deemed to have sufficient catalytic activity to effect the desired result hereunder is intended to be comprehended hereby.

The hydrogenation may be carried out according to any known protocols and methods, and using conventional apparatus. For example, such may be done in a tubular or a stirred tank reactor. Effective reaction temperatures may range from about 50° C. to about 400° C. or higher, preferably from about 100° C. to about 300° C., for a period of from about 1 hour or less to about 4 hours or longer, with the longer times being in some embodiments employed in conjunction with the lower temperatures. Reaction pressures may range from 15 psig to about 3000 psig or greater. In certain preferred and non-limiting embodiments, mild temperatures and low pressures may be generally considered desirable in promoting acceptable catalyst performance and lifetime, as well as product stability. The amount of hydrogenation catalyst used is dependent on the particular hydrogenation catalyst employed and may range, in certain non-limiting embodiments, from about 0.01 weight percent or less to about 10 weight percent or greater, based on the total weight of the starting materials.

### Uses

Applications of the invention may include a wide variety of formulations and products. These include, but are not limited to, kitchen cleaners, cleaners for triglycerides, cross-linked triglycerides, or mixtures thereof, cleaners for mineral-oil type soils, hydrotropes for formula stabilization, surfactant for ultra-concentrate formulas, self-hydrotroping surfactants for enhanced formula stabilization with surfactant activity, general cleaners, pre-wash spotting agents, pre-wash concentrates, detergents, hard surface cleaning formulations.

In alternative embodiments, the surfactants of Formulae (I) and (II) find use in polyurethanes, epoxies, thermoplastics, paints, emulsions for paints and coatings, such as poly(acrylates), coatings, metal products, agricultural products including herbicides and pesticides, mining products, pulp and paper products, textiles, water treatment products, flooring products, inks, colorants, pharmaceuticals, personal care products, lubricants, and a combinations of these.

In preparing these and other types of formulations and products, the alcohol alkoxylate may contribute to or enhance a desirable property, such as surfactancy, detergency, wetting, re-wetting, foam reduction, additive stabilization, latex stabilization, as an intermediate in reactions involving ester formation or urethane formation, drug delivery capability, emulsification, rinsing, plasticization, reactive dilution, rheology modification, suspension, pseudoplasticization, thickening, curing, impact modification, lubrication, emulsification and micro-emulsification, a combination thereof, or the like.

Examples of these applications include utility of compositions of Formulae (I) and (II) as surfactants in general; as surfactants for household and commercial cleaning; as surfactants for the cleaning of triglyceride or cross-linked triglyceride soils, as hydrotropes for enhancing formula stability, as self-hydrotroping surfactants to eliminate or reduce hydrotropes from formulas, pre-wash spotters, laundry, ultra-concentrated laundry formulations ultraconcentrated hard-surface cleaning formulations, ultraconcentrated dilutable surfactants, as surfactants for imparting freeze-thaw stability in paints and coatings, as surfactants for imparting freeze-thaw stability for pigment dispersion, as surfactants in mechanical cleaning processes, as surfactants for use in cleaning kitchens or industrial kitchens, as surfactants for cleaning areas with cross-linked triglycerides such as grills, kitchen ware, stoves, and walls, as reactive diluents in casting, encapsulation, flooring, potting, adhesives, laminates, reinforced plastics, and filament windings; as coatings; as wetting agents; as rinse aids; as defoam/low foam agents; as spray cleaning agents; as emulsifiers for herbicides and pesticides; as metal cleaning agents; as suspension aids and emulsifiers for paints and coatings; as mixing enhancers in preparing microheterogeneous mixtures of organic compounds in polar and non-polar carrier fluids for agricultural spread and crop growth agents; as surfactants for agricultural adjuvants, as stabilizing agents for latexes; as microemulsifiers for pulp and paper products; and the like. In one non-limiting embodiment, compositions utilizing the alkoxylates may include microemulsions used for organic synthesis and/or cleaning, formation of inorganic and organic particles, polymerization, and bio-organic processing and synthesis, as well as combinations thereof. In other non-limiting embodiments, the alkoxylates described herein may serve to dilute higher viscosity epoxy resins based on, for example, bisphenol-A, bisphenol-F, and novolak, as well as other thermoplastic and thermoset polymers, such as polyurethanes and acrylics. They may also find use in rheology modification of liquid systems such as inks, emulsions, paints, and pigment suspensions, where they may also be used to impart, for example, enhanced biodegradability, pseudoplasticity or thixotropic flow behavior. In these and other uses the alkoxylates may offer good and, in some cases, excellent performance, as well as relatively low cost.

As noted above, the surfactants of the invention are useful as agricultural adjuvants. In particular, the surfactants can enhance the activity of several different classes of herbicides on a wide variety of weeds. Non-limiting examples of such herbicides include: glyphosates, such as glyphosate isopropylamine; auxins and pyridines, such as 2,4-dichlorophenoxyacetic acid (2,4-D), clopyralid, picloram, etc.; cyhalofop, haloxyfop and other fops as well as cyclohexandiones; sulfonamides, sulfonylureas, imidazalinones; and HPPD inhibitors such as mesotrione.

The amount of optional ingredients effective for achieving the desired property provided by such ingredients can be readily determined by one skilled in the art.

### EXAMPLES

The following examples are for illustrative purposes only and are not intended to limit the scope of the present invention.

### Example 1

Exemplary surfactants of the present invention can be made by the following protocol: All alkoxylation feed and digest steps are performed at about 130° C. All alkoxylations are performed with an approximate oxide feed rate of about 5.0 grams/minute with a subsequent digest/cookout time (for each step) of at least 4 hours.

A 2-ethyl hexanol (“2EH”) alkoxylate can be produced by taking of 2-ethyl hexanol and catalyzing with grams flake (85%) KOH, and drying under a vacuum 5 mm Hg at 100° C. for about 30 minutes or until the water level is below 1000 ppm. The material is alkoxylated by feeding propylene oxide in an autoclave to result in an intermediate 2EH(PO)ₓ alkoxylate. After a suitable cookout at 130° C., the intermediate is subsequently ethoxylated by feeding ethylene oxide to result in an intermediate 2EH(PO)ₓ(EO)_{y}. After a suitable cookout at 130° C., the material is removed from the reactor and neutralized with acetic acid to a pH range of 4-8 (as a 10% aqueous solution) to afford the product.

A surfactant made substantially according to the protocol described above was produced by taking 813 grams of 2-ethyl hexanol catalyzing with 2.07 grams flake (85%) KOH, drying under a vacuum 5 mm Hg at 100° C. for 30 minutes hours until the water level was below 1000 ppm. The material was alkoxylated by feeding 725 grams propylene oxide in an autoclave to result in an intermediate 2EH(PO)₂ alkoxylate. After a suitable cookout at 130° C. the material was subsequently ethoxylated by feeding 1100 grams of ethylene oxide to result in an intermediate 2EH(PO)₂(EO)₄. After an appropriate cookout at 130° C., the material was removed from the reactor and neutralized with acetic acid to a pH range of 4-8 (as a 10% aqueous solution).

### Examples 2-8

Surfactants made substantially according to the protocols described above in Example 1 were made and recited in TABLE 1.

TABLE 1

| | Compound | 2EH feed | KOH feed | PO feed | EO feed |
|---|---|---|---|---|---|
| Example 1 | 2EH(PO)₂(EO)₄ | 813 g | 2.07 g | 725 g | 1100 g |
| Example 2 | 2EH(PO)₃(EO)_{6.8} | 8823 g | 1.96 g | 1105 g | 1905 g |
| Example 3 | 2EH(PO)_{5.5}(EO)₈ | 1051 g | 3.66 g | 2495 g | 2965 g |
| Example 4 | 2EH(PO)₉(EO)₉ | 561 g | 2.77 g | 2245 g | 1710 g |
| Example 5 | 2EH(PO)₁₁(EO)₁₁ | 415 g | 2.44 g | 2025 g | 1555 g |
| Example 6: | 2EH(PO)₅(EO)₃ | 1.0 mole | 0.58 wt % | 5.0 mole | 3.0 mole |
| Example 7 | 2EH(PO)₅(EO)₆ | 1.0 mole | 0.58 wt % | 5.0 mole | 6.0 mole |
| Example 8 | 2EH(PO)₅(EO)₉ | 1.0 mole | 0.58 wt % | 5.0 mole | 9.0 mole |

### Example 9

Ten gallons of a mixed internal octene/octane stream was obtained from the Dow Chemical Company polyolefins R&D group. The composition of this stream (in percentage) was approximately

1-octene: 21.2

Trans-3-me-3-heptene: 1.3

Trans-4-octene 1.7

(trans-3-octene, cis-3-me-3 heptene, trans-3-me-2-heptene, cis-3-octene, cis-4-octene): 13.6%

Trans-2-octene: 6.3%

Cis-3-me-2-heptene: 2.6%

Cis-2-octene: 4.1%

Isopar-E (Isooctane alkane) 49%

Hydroformylation of C8 Olefin Stream—2 Gallon Reactor Runs to prepare C9 Aldehyde

Example Hydroformylation Run

Catalyst charge/reaction mixtures were prepared and transferred under nitrogen atmosphere. The Octene/Isopar™ E mixture was sparged with nitrogen for˜15 minutes before use. A catalyst charge was prepared from:

3.2727 grams Rh(CO)2(acac)

161.4 grams Doverphos

2606 grams Octene/Isopar™ E, D-621 (ID#283256)

A 2 gallon reactor was inerted with nitrogen and charged with 2769 grams of the above catalyst solution and an additional 1812 grams Octene/Isopar™ E. The reactor was pressured/vented 2 times to 75 psig with 1:1 H2/CO then heated to 90° C. Upon reaching 60° C. the reactor was pressured to 500 psig with 1:1 H2/CO and the pressure maintained at 500 psig with 1:1 H2/CO for the duration of the run.

After 8 hours of reaction the reactor was cooled and left under a syngas atmosphere overnight.

### Hydrogenation of Mixed C9Aldehyde/Isopar Mixture

Approximately 13 kilograms of crude Mixed C9 Aldehyde/Isopar was hydrogenated to Mixed C9 Alcohol/Isopar™. The liquid phase hydrogenation took place over a three day period using a continuous fixed bed operation with Engelhard™ Ni-3288 E 1/16×3F catalyst. The feed tank charge was the composite of three, two-gallon hydroformylation batch runs. A total of 12,707 grams was charged to the feed tank. Crude mixed C9 Aldehyde/Isopar™ was fed directly to the hydrogenation without removal of the Rh/Doverphos which had been used in the hydroformylation of the Octene/Isopar™ purge stream.

The hydrogenation reactor was configured with a feed preheater and a 1″ by 4 ft reaction tube (400 cc) configured as an upflow, packed-bed column, having liquid as the continuous phase with the aldehyde being the limiting reactant and saturated with hydrogen gas. The reactor catalyst charge was 309 grams of nickel 3288 E, 1/16×3F Engelhard™ lot No. DM00431. The catalyst was in the reduced and stabilized form. One millimeter glass beads were used in the inlet and outlet of the tube reactor; the glass beads were covered with glass wool.

The aldehyde/Isopar™ was fed at˜730 grams/hr and hydrogen flow was maintained at 36 liter/hr. keeping hydrogen in molar excess. The reactor preheater was set at 90° C. and the reactor heater set at 100° C. The typical or average temperature rise up the reactor tube was from 90 to 120° C. Pumping of the aldehyde/alcohol/Isopar™ continued in recycle mode for 36 hours (˜2.2 passes), then the reactor product was diverted to the product tank for a final pass which required 17.2 hours. Total passes through the reactor was approximately three.

### PURIFICATION (Post-Hydrogenation of the Crude Nonanol/Octane Stream)

To a Buchii™ R-220 3-gallon rotary evaporator distillation flask was added 6.0 L of a crude˜50/50 wt % solution of C9 alcohol in Isopar™ E containing residual hydroformylation ligand. Rotation of distillation flask was started at 89 RPM and 560 bar. The water bath was heated to 80° C. When the water bath reached the desired temperature, the pressure was lowered in 100 bar increments to 50 bar to remove˜3.0 L of Isopar™ E. The residual C9 alcohol was then distilled away from the residual hydroformylation ligand in 500 ml batches at 1-5 torr vacuum.

Alkoxylation of the alcohol to give C9(BO)1(EO)7

All alkoxylation feed and digest steps were performed at 130 C. All alkoxylations were performed with an approximate oxide feed rate of 5.0 grams/minute with a subsequent digest/cookout time (for each step) of at least 4 hours. An alkoxylate was produced by taking 1364 grams of purified nonanol (from above), catalyzing with 3.35 grams flake (85%) KOH, drying under a vacuum 5 mm Hg at 100 C for 30 minutes hours until the water level was below 1000 ppm. The material was alkoxylated by feeding 690 grams butylene oxide in an autoclave to result in an intermediate C9(BO)1 alkoxylate. After flushing and sampling, 3193 grams remained in the reactor. The material was subsequently ethoxylated by feeding 1255 grams of ethylene oxide to result in an intermediate C9(BO)1(EO)3 with a cloud point of <10 C. After flushing and sampling 3409 grams remained in the reactor, The material was further ethoxylated with 400 grams of ethylene oxide a to result in an intermediate C9(BO)1(EO)4 with a cloud point of <10 C. After flushing and sampling 3674 grams remained in the reactor. This material was further ethoxylated with 380 grams of ethylene oxide to result in an intermediate C9(BO)1(EO)5 with a cloud point of 21.4 C. After flushing and sampling, 3674 grams remained in the reactor. Further ethoxylation with 260 grams of ethylene oxide resulted in C9(BO)1(EO)6 with a cloud point of 38.5 C. After flushing and sampling, 3197 grams remained in the reactor (704.1 grams of this material was removed for subsequent performance testing) The remaining material was ethoxylated with 285 grams of ethylene oxide to result in approximately 3482 grams C9(BO)1(EO)7 with a cloud point of 53.9 C. The material was removed from the reactor, neutralized with acetic acid to a pH range of 4-8 (as a 10% aqueous solution).

### Example 10

### C9(PO)4(EO)8

The C9 alcohol prepared in Example 9 was used as the starting alcohol.

All alkoxylation feed and digest steps were performed at 130° C. All alkoxylations were performed with an oxide feed rate of approximately 5.0 grams/minute with a subsequent digest/cookout time (for each step) of at least 4 hours. An alkoxylate was produced by taking 500.2 grams of purified nonanol (from above), catalyzing with 2.64 grams flake (85%) KOH, drying under a vacuum 5 mm Hg at 100° C. for 30 minutes hours until the water level was below 1000 ppm. The mass of alcohol after flashing and sampling was 472.15 grams. 301.9 grams of fresh, dry C9 alcohol was added to the catalyzed alcohol, and sampled for catalyst verification. The final alcohol weight, after sample extraction was 752 g. The alcohol was subsequently propoxylated with 1220 grams of PO. The material was then ethoxylated with 1265 grams of EO to produce a C9(PO)4(EO)5.5 with a cloud point of 31.0 C. A sample of 61.1 grams was removed from the reactor for testing purposes. The remaining material was ethoxylated with 290 grams of EO to produce a C9(PO)4(EO)6.8 with a cloud point of 43.0° C. A sample of 167 grams was removed from the reactor for analysis. The remaining material was ethoxylated with 265 grams of EO to result in a final C9(PO)4(EO)8 with a mass of 3565 grams and a cloud point of 55.3° C. The material was removed from the reactor, neutralized with acetic acid to a pH range of 4-8 (as a 10% aqueous solution).

### Example 11

### C9(PO)4(EO)6

The C9 alcohol prepared in Example 10 was used as the starting alcohol. Alkoxylation conditions were similar to those used in Example 10, except that the molar ratio of reactants was 1 mole C9 alcohol, 4 moles PO, and 6 moles EO, with a catalyst (KOH, s) level of approximately 0.5 weight %

### Example 12

### Testing

### Cleaning of Cross-Linked Triglycerides

Test panels coated with mixtures of triglycerides and cross-linked triglycerides were prepared and evaluated using the following procedure. Cobalt Naphthenate was used as a catalyst to accelerate the oxidation of vegetable oil to give a hard varnish. Carbon black is added to the varnish to enable easy visual comparison of the ability to clean the cross-linked triglyceride from the surface.

Substrate Panels: White Vinyl Floor Tile: Tarket Corporation Azrock™ VS304-3 (6913) cut to 4¼×4¼ inch (to fit the Gardner Linear Motion Scrubber).

Soil Formulation: 100 grams Canola Oil (Food Grade, 100%, Kroger Co. Cincinnati, Ohio 45202); 2 grams Acetylene Carbon Black (Cat #39724, Alfa Aesar; Surface area=74 sq m/g); 20 grams drying agent: Cobalt Naphthenate Solution (Aldrich Cat #54,457-4, CAS #61789-51-3) (Comes as a 6% solution in petroleum solvent); Oven: Convection oven set at 160° F.

Scrubbing Tester: Gardco washability & Wear Tester; Linear Motion Test Equipment; Model D12-V Cat #WA-2164 (Paul N. Gardner Company, Inc. 316 N.E. First Street, Pompano, Beach, Fla. 33060.

Sponges: “Do-It” Cellulose Sponge, 1⅝ in thick, cut to 3″×4″. Manufactured by Bloch/New England for HWI, Fort Wayne, Ind. 46801;

Paint Brushes: Economy Chip Brush 1 Inch. www.lgsourcing.com Model #0106; Item #103407 obtained from Lowes, Inc. L. G. Sourcing, Inc. P.O. Box 1535 North Wilkseboro, N.C. 28659

Reflectance Meter: HunterLab ColorQuest XE

### Procedure

Prepare a stock solution by mixing 98 grams of canola oil (food grade) with 2.0 g acetylene-based carbon black (Cat #39724, Alfa Aesar; Surface area=74 sq m/g). Mix with a disperser at 2000 rpm for 15 min. (We used a Caframo Model BDC 3030 with a 0.50 dispersing blade).

Add 20.0 grams Cobalt Naphthenate Solution and mix well (by hand, using a glass stir rod).

Place 1.6 grams soil per 4×4 inch tile.

Paint to a thin film using a clean 1-inch economy brush. Use several strokes to get to an even coating. Use a clean, dry brush for each application. (After the application, each brush can be cleaned with acetone, dried, and then re-used).

Place in a convection oven set at 160 F for 16 hours. We place the panels in an oven at 4:00 p.m, and then remove the panels at 8:00 a.m. the next morning. Let the panels cool for 1 hour.

Use the panels within 10 days

Place a panel in the Gardner Scrubber

Prepare 500 mL 1% solution of surfactant in water. (We use 5.0 grams surfactant diluted to 500 mL water).

Prepare a sponge by rinsing several times in cold tap water. Completely squeeze out the sponge by hand.

The sponge may be used for up to >50 tests, or until the sponge looses its elasticity (when the sponge does not recover its original shape after being squeezed). After each test, rinse out the sponge 15-20 times (by repeated sorption and squeezing) until there is no noticeable surfactant solution left (usually indicated by a lack of foam).

Pour 500 mL of surfactant solution into a beaker

Place the sponge into the beaker—allowing it to soak up as much surfactant as possible.

Place the sponge into the Gardner Scrubber.

Pour the remaining liquid (approx 400 mL) over the test panel in the scrubber. There should be enough liquid to just cover the test panel.

Program the scrubber to perform 120 back-and-forth strokes (for a total of 240 linear strokes). We define each back-and-forth stroke as “1 stroke”

Remove the panel rinse with tap water

Clean the Gardner scrubber by rinsing with tap water.

Clean the sponge by squeezing it out under tap water 20-30 times until clean.

Remove excess water from the sponge by squeezing as much liquid out as possible.

After the panel is dry, either take pictures (for visual comparison of cleaning) or measure the reflectance using the Xyy mode of a Hunter Colorimeter. Alternatively, the mean gray value can be obtained by taking a picture of the tiles and processing computer image with ImageJ™ software, which is distributed freely by the National Institute of Health (nih.gov).

Table 2 shows the cleaning of cross-linked triglycerides using 1.0% aqueous solutions, with 120 back-and forth strokes using the procedure above. Several competitive offsets were used as comparison. The data shows that 2EH(PO)5(EO)6 (Example 7) performs as well as NP-9, whereas other Surfactants did not work as well. Note that higher arbitrary gray values correspond to better cleaning.

TABLE 2

| Sample (1% by weight in water) | Arbitrary Gray Value |
|---|---|
| C12-14(EO)5 (Comparative) | 68 |
| NP-9 (Comparative) | 177 |
| Example 7 2EH(PO)5(EO)6 | 179 |
| Example 8 2EH(PO)5(EO)9 | 128 |
| Example 9 C9(PO)4(EO)8 | 121 |

Table 3 shows the cleaning of cross-linked triglycerides using 0.5% aqueous solutions, with 120 back-and forth strokes using the procedure above. Several competitive offsets were used as comparison. The data shows that 2EH(PO)5(EO)6 performs as well and NP-9, whereas other commercially available surfactants do not perform as well. Note that higher arbitrary gray values correspond to better cleaning.

TABLE 3

| Sample (0.5% by weight in water) | Arbitrary Gray Value |
|---|---|
| NP-9 (Comparative) | 108 |
| Example 7 2EH(PO)5(EO)6 | 106 |
| C8-16(PO)2.5(EO)5 (Comparative) | 48 |
| Lutensol ™ XP-50 (Comparative) | 59 |
| Lutensol ™ XL-70 (Comparative) | 53 |
| Tomadol ™ 901 (Comparative) | 56 |

### Cleaning of Cross-Linked Petroleum Grease

The same procedure used above for cross-linked triglycerides was used, except that 1-octadecene was used instead of Canola Oil.

Table 4 shows the cleaning of cross-linked 1-octadecene using 2EH(PO)5(EO)8 vs. NP-9 and Lutensol XP-70. The data shows that the 2EH alkoxylate is equivalent to Tergitol NP-9 in cleaning cross-linked mineral oil.

TABLE 4

| Sample (1% by weight in water) | Arbitrary Gray Value |
|---|---|
| NP-9 (Comparative) | 95 |
| Example 3 2EH(PO)5.5(EO)8 | 136 |
| Lutensol ™ XP-70 (Comparative) | 120 |

### Biodegradation

The biodegradability of the alkoxylates according to the invention are tested by exposing the alkoxylates to microorganisms derived from activated sludge obtained from a municipal sewage treatment plant under aerobic static exposure conditions, using standard OECD 301 F methodology. OECD 301 F refers to the Organization for Economic Cooperation and Development Guidelines for the Testing of Chemicals, “Ready Biodegradability: Manometric Respirometry Test,” Procedure 301 F, adopted 17 Jul. 1992, which is incorporated herein by reference in its entirety.

### Aquatic Toxicity

The study procedures and test methods were based on the recommendations of the following guidelines:

Organization for Economic Cooperation and Development (OECD): OECD Guidelines for the Testing of Chemicals, “Freshwater Alga and Cyanobacteria, Growth Inhibition Test”, Procedure 201, adopted 23 Mar. 2006; European Economic Community (EEC): Commission directive 92/69/EEC of 31 Jul. 1992, Methods for the determination of ecotoxicity, C.3., “Algal Inhibition Test”.

OECD Guidelines for the Testing of Chemicals, “Freshwater Alga and Cyanobacteria, Growth Inhibition Test”, Procedure 201, adopted 23 Mar. 2006; European Economic Community (EEC): Commission directive 92/69/EEC of 31 Jul. 1992, Methods for the determination of ecotoxicity, C.3., “Algal Inhibition Test”.

Data from the biodegradation and aquatic toxicity tests is shown in TABLE 5.

TABLE 5

| | | Fresh Water | | |
|---|---|---|---|---|
| | OECD 301F | algal growth | 48-hour Acute | |
| | Biode- | inhibition test with | Toxicity to | |
| | gradation, | Desmondesmus | Daphna magna | |
| Compound | % | subspicatus ErC50/0-3 | (EC50-50 hour) | |
| Example 6 | 74 | 31.9 mg/L | 33.6 | mg/L |
| 2EH(PO)5(EO)3 | | | | |
| Example 8 | 79 | 97.7 mg/L | >100 | mg/L |

| | | | |
|---|---|---|---|
| 2EH(PO)5(EO)9 | | | |
| Example 9 | 73 | 21 | 6.2 |
| C9(BO)1(EO)7 | | | |
| Example 10 | 70 | 26 | 29.2 |
| C9(PO)4(EO)8 | | | |

### Formula Stability:

When mixed with dodecyl benzene sulfonic acid (sodium salt), and sodium citrate in water, the surfactants of the present invention show enhanced formula stability relative to conventional surfactants. This is shown below in TABLE 6, stability of surfactants of the invention when mixed with formulas containing LAS (dodecyl benzene sulfonic acid, sodium salt), sodium citrate, and water, which shows that the surfactant of Example 5 is stable in cleaning formulations, relative to conventional surfactants:

TABLE 6

| | 2EH(PO)11(EO)11 | NP-9 | TERGITOL ™ 15-S-9 |
|---|---|---|---|
| Formula Composition | (Example 5) | (Comparative) | (Comparative) |
| 15% LAS/0% Na Cit | S | U | U |
| 15% LAS/1% Na Cit | S | U | U |
| 15% LAS/2% Na Cit | S | U | U |
| 15% LAS/4% Na Cit | U | U | U |

S = Stable;
U = Unstable

### Fundamental Surfactant Properties:

A) Ross-Miles Foam Height Test: This test is carried out according to the protocol of ASTM D1173.

B) Surface Tension and Critical Micelle Concentration (CMC) Measurement. For this test the surface tension of a surfactant-water solution is measured while incrementally adding the surfactant to de-ionized water. Results are measured in terms of dyne/centimeters using a Wilhelmy plate. Results are recorded versus surfactant concentration. The Critical Micelle Concentration is the point at which an increase in surfactant concentration no longer results in a change in surface tension.

C) Pour Point Test: This test is carried out according to the protocol of ASTM Test D97.

D) Gel range: Ten surfactant solutions are made using 0%, 10%, 20%, 30%, 40%, 50%, 60%, 70%, 80%, 90% water. If the resulting solutions form a gel, and do not pour, then they are identified as a “gel”. The test is run at 23 C.

Table 7 shows the gel range and pour points of surfactants of the invention relative to other benchmark surfactants:

TABLE 7

| | Pour | | | | | | | | | |
|---|---|---|---|---|---|---|---|---|---|---|
| | Point | Gel Range, Percent Surfactant in Water at 23 C. | | | | | | | | |
| Sample | ° F. | 10% | 20% | 30% | 40% | 50% | 60% | 70% | 80% | 90% |
| 2EH(PO)3(EO)7 | 50 | L | L | L | L | L | L | L | L | L |
| Example 2 | | | | | | | | | | |
| 2EH(PO)5.5(EO)8 | 44 | L | L | L | L | L | L | L | TL | L |
| Example 3 | | | | | | | | | | |
| 2EH(PO)9(EO)9 | 37 | L | L | L | TL | G | G | G | G | L |
| Example 4 | | | | | | | | | | |
| 2EH(PO)11(EO)11 | 36 | L | L | L | G | G | G | G | G | L |
| Example 5 | | | | | | | | | | |
| Tergitol ™ NP-9 | 30 | L | L | L | L | L | G | G | G | L |
| (Comparative) | | | | | | | | | | |
| Tergitol ™ 15-S-9 | 44 | L | L | L | L | G | L | L | L | L |
| (Comparative) | | | | | | | | | | |
| Neodol ™ 25-7 | 80 | L | L | L | G | G | G | G | G | L |
| (Comparative) | | | | | | | | | | |
| Neodol ™ 1-9 | 64 | L | L | L | L | G | G | G | L | L |
| (Comparative) | | | | | | | | | | |
| TDA-9 | 68 | L | L | L | L | L | L | G | G | L |
| (Comparative) | | | | | | | | | | |
| Tomadol ™ 900 | 38 | L | L | L | L | G | L | L | L | L |
| (Comparative) | | | | | | | | | | |
| Tomadol ™ 901 | 38 | L | L | L | L | L | G | G | G | L |
| (Comparative) | | | | | | | | | | |

L = Liquid;
G = Gel,
TL = Thick Liquid.

Table 8 shows the critical micelle concentration vs. the degree of propoxylation for a series of 2-Ethyl Hexanol Alkoxylates. Generally, better surfactant efficacy is obtained with lower CMC's. Propoxylation beyond about 5.5 moles of PO results in products that are not biodegradable. A critical balance between low CMC and biodegradability is obtained with a degree of propoxylation of 5.5 (or from about 4-5.5)

Table 8 shows the surface tension (0.1 wt % in water) vs. the degree of propoxylation for a series of 2-Ethyl Hexanol Alkoxylates. Generally, better surfactant efficacy is obtained with lower surface tensions. Propoxylation beyond about 5.5 moles of PO results in products that are not biodegradable. A critical balance between low surface tension and biodegradability is obtained with a degree of propoxylation of 5.5 (or from about 4-5.5).

TABLE 8

| | Critical Micelle | |
|---|---|---|
| Sample | Concentration | Surface Tension |
| Example 1 2EH(PO)2(EO)4 | 3300 | 35 |
| Example 2 2EH(PO)3(EO)6.8 | 2400 | 32 |
| Example 3 2EH(PO)5.5(EO)8 | 1750 | 31 |
| Example 4 2EH(PO)9(EO)9 | 400 | 30 |
| Example 5 2EH(PO)11(EO)11 | 300 | 30 |

Table 9 shows the Ross-Miles foam (0 sec, 360 sec) of the invention, relative to conventional surfactants.

TABLE 9

| | Ross Miles Foam Height, millimeters | |
|---|---|---|
| Sample | Initial | 5 Minutes |
| Example 1 2EH(PO)2(EO)4 | 110 | 5 |
| Example 2 2EH(PO)3(EO)6.8 | 115 | 5 |
| Example 3 2EH(PO)5.5(EO)8 | 45 | 0 |
| Example 4 2EH(PO)9(EO)9 | 50 | 5 |
| Example 5 2EH(PO)11(EO)11 | 75 | 15 |
| NP-9 (Comparative) | 145 | 35 |
| PAE-7 (Comparative) | 105 | 100 |

### Efficacy in Agricultural Applications

Efficacy of Examples 7 and 8 as adjuvants in formulated herbicides is compared to commercially available herbicide packages. Greenhouse field testing is completed. A 480 g ae/L (acid equivalent per liter) formulation of glyphosate isopropylamine with no adjuvants is added to spray vials. These aliquots are diluted to a final volume of 60 ml with tap water, and appropriate amounts of adjuvants are added to the spray solution. The Examples 7 and 8 series are tested at 0.25% v/v in the final spray solution. Treatment rates are: 200, 400, and 600 g ae/ha (“ha” means hectare) and each treatment is replicated three times. Treatments are applied with a tracksprayer. The sprayer utilizes an 8002E spray nozzle, spray pressure of 262 kPa pressure and speed of 2.2 mph to deliver 140 L/Ha. The nozzle height is 46 cm above the pots. Percent visual injury assessments are made at 18 DAA (days after application) on a scale of 0 to 100% as compared to the untreated control plants (where 0 is equal to no injury and 100 is equal to complete death of the plant). Results are shown in Table 10 as % Control of Sicklepod with Glyphosate compared to commercial herbicides.

TABLE 10

| Formulation | 200 G/ha | 400 G/ha | 600 G/ha |
|---|---|---|---|
| Control | 16.7 | 63.3 | 75.0 |
| Example 8 | 31.7 | 73.3 | 75.0 |
| Example 7 | 66.7 | 81.7 | 98.3 |
| DURANGO ® | 53.3 | 80.0 | 92.5 |
| (Comparative) | | | |
| WEATHERMAX ® | 58.3 | 83.3 | 91.7 |
| (Comparative) | | | |

It is understood that the present invention is not limited to the embodiments specifically disclosed and exemplified herein. Various modifications of the invention will be apparent to those skilled in the art. Such changes and modifications may be made without departing from the scope of the appended claims.

Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1.-16. (canceled)

17. A method of removing cross-linked triglycerides from a surface, comprising
applying to said surface a cleaning composition comprising:
at least one nonionic surfactant represented by formula (I):
R¹—O—(CH₂CH(R²)—O)ₓ(CH₂CH₂O)_{y}—H (II)
wherein:
x is about 5;
y is about 3, 6, 9, or 11;
R¹ is 2-ethyl hexanol; and
R² is CH₃ or CH₂CH₃.

18. The method of claim 1, wherein the surface is a textile.

19. Use of a nonionic surfactant represented by formula (II):
R¹—O—(CH₂CH(R²)—O)ₓ(CH₂CH₂O)_{y}—H (II)
wherein:
x is about 5;
y is about 3, 6, 9, or 11;
R¹ is 2-ethyl hexanol; and
R² is CH₃ or CH₂CH₃,
as an agricultural adjuvant.

20. The method of claim 1, wherein the surface is tile.
